(19) United States
(12) Patent Application Publication
(10) Pub. No.: US 2026/0348273 A1
(43) Pub. Date: Nov. 5, 2026

(54) METHOD FOR IMPROVED DRILLING AND BLASTING IN OPEN CUT MINES

(71) Applicant: Technological Resources Pty. Ltd, Melbourne (AU)

(72) Inventors: Ozan Perincek, Melbourne (AU); Ross P. Williams, Melbourne (AU); Claudia Haugg, Melbourne (AU); Michael Thomas, Melbourne (AU); Michael Beswick, Melbourne (AU)

(73) Assignee: Technological Resources Pty. Ltd, Melbourne (AU)

(21) Appl. No.: 19/120,370

(22) PCT Filed: Oct. 11, 2023
(86) PCT No.: PCT/AU2023/051001

§ 371 Date: Apr. 11, 2025

(30) Foreign Application Priority Data
Oct. 11, 2022 (AU) 2022902980

(51) Int. Cl. E21B 49/00; G01N 15/02; G01N 33/24

(52) U.S. Cl. CPC E21B 49/003; G01N 15/02; G01N 33/24; E21B 2200/20; E21B 2200/22

(57) ABSTRACT

A method to improve accuracy of hole-by-hole ground hardness predictions for a bench (“bench-below”), being a bench that is below a current bench of an open cut mine. The method includes generating measure while drilling (MWD) data, whilst performing blast hole drilling of the current bench. The MWD data includes air pressure data indicating air pressure measurements from an air pressure sensor, and torque data indicating measurements of torque that opposes rotation of a drill bit performing the blast hole drilling. The method also includes determining occurrence of fallback or malfunction of a pressurized air delivery arrangement during drilling based upon the air pressure data and the torque data and discarding some or all MWD data collected during the occurrence of the fallback or malfunction of the pressurized air delivery arrangement, whilst generating the hole-by-hole ground hardness predictions. Determining the occurrence of fallback or malfunction during drilling may include monitoring the air pressure data and the torque data to identify an increase in air pressure whilst at the same time identifying an increase in torque.

### RELATED APPLICATIONS

The present application is a national stage application (under 35 U.S.C. § 371) of PCT/AU2023/051001, filed Oct 11, 2023, which claims priority from Australian Patent Application No. 2022902980, filed 11 Oct. 2022, the contents of each of which is hereby incorporated in thier entirety by reference.

### TECHNICAL FIELD

The present invention relates to a method and system for conducting blasting in an open cut mine in a controlled manner to produced desired blasting outcomes whilst avoiding deleterious effects such as under-blasting and over-blasting.

### BACKGROUND ART

Any references to methods, apparatus or documents of the prior art are not to be taken as constituting any evidence or admission that they formed, or form part of the common general knowledge.

Mining operations involve blasting ground that has been ascertained to contain mineral deposits to generate blasted material that is then loaded by loading vehicles into haul trucks for subsequent processing by plant such as crushers and blending stations.

It will be realized that depending on the blast pattern and explosive parameters that are applied, the results of blasting will vary significantly. For example, use of too much explosive, i.e. “overblasting” may cause the resulting blasted rock to be of dimensions that are too small to be efficiently processed by customers so that a “lump premium” for suitably dimensioned blasted rock is no longer payable. Alternatively, use of too little explosive may result in blasted rock fragments that are too large to be readily loaded or processed by machines such as crushers. Processing large rock fragments requires the expenditure of additional time and energy relative to processing rock fragments that fall within a desired target range of dimensions so that the associated overall tons per hour production rate falls.

A technical problem that the present Inventors have become aware of is that information gleaned from processing Measure While Drilling data that is received from drilling rigs may be inconsistent or even contradictory so that it is difficult to accurately ascertain a correct understanding of the physical properties of the area in which it is desired to perform blasting. Consequently, despite best efforts the blast pattern and the parameters of the explosive that are used may result in unexpected and undesirable outcomes associated with over-blasting and under-blasting.

For example, in international patent publication WO 2022/016207 A1, filed on 29 Jun. 2021 by Orica Australia Pty Ltd, there is described a method for designing a pattern of blast holes by mapping blastability labels from geographical data obtained by Measure While Drilling (MWD). However, the method that is described may be prone to producing less than desirable blast outcomes due to inaccuracies in some of the geographical data that is obtained.

In the Applicant's earlier international patent application no PCT/AU2021/051512 Method and system for automated rock recognition, MWD data is processed, using techniques such as clustering and principal component analysis to derive a visualization of the distribution of characteristic measures and rock hardness for each cluster. If the MWD data at a depth is identified as an outlier, then all MWD data for that hole at that depth may be removed. Whilst the methods described in PCT/AU2021/051512 are highly useful for creating visualization of the distribution and hardness of various types of rock, it remains the case that there is a need for better reducing the likelihood of unexpected and undesirable outcomes associated with over-blasting and under-blasting. Substantial research, experimentation, trial and error, and lateral thinking have been exercised by many technical staff, including engineers and scientists, of mining companies over a number of years with the objective of providing a technical solution to the problem that has been discussed. As will be explained, the present Inventors have conceived of, and reduced to practice, a method and system for improving drill and blast that takes into account specific physical characteristics of the geographical area in which blast patterns are deployed.

### SUMMARY

According to a first aspect, there is provided a method to improve accuracy of hole-by-hole ground hardness predictions for a bench (“bench-below”) that is below a current bench, the method comprising:
generating measure while drilling (MWD) data, whilst performing blast hole drilling of the current bench, wherein the MWD data includes,
   air pressure data indicating air pressure measurements from an air pressure sensor, and
   torque data indicating measurements of torque opposing rotation of a drill bit performing the blast hole drilling;
determining occurrence of fallback or malfunction of a pressurized air delivery arrangement, during drilling based upon the air pressure data and the torque data; and
   discarding some or all MWD data collected during the occurrence of the fallback or malfunction of the pressurized air delivery arrangement, whilst generating the hole-by-hole ground hardness predictions.

In an embodiment the determining of the occurrence of fallback or malfunction of a pressurized air delivery arrangement during drilling includes monitoring the air pressure data and the torque data to identify an increase in air pressure whilst at the same time identifying an increase in torque.

In an embodiment the determining of the occurrence of fallback or malfunction of the pressurized air delivery arrangement during drilling is performed by a fallback model that is responsive to the air pressure data and the torque data, wherein the fallback model generates a fallback detected signal that indicates the occurrence of the fallback or malfunction of the pressurized air delivery arrangement.

In an embodiment the method includes generating the ground hardness predictions by applying the MWD data to a pre-trained deep learning (DL) model wherein the pre-trained deep learning model is responsive to the MWD data to produce a deep learning hardness prediction.

In an embodiment the method includes providing a Rate of Penetration (ROP) model that is responsive to ROP data of the MWD data, wherein the ROP model produces ROP hardness data indicating hardness of the current hole.

In an embodiment the method includes providing a Specific Energy of Drilling (SED) model, wherein the SED model is configured to produce SED Hardness data in response to data of the MWD data.

In an embodiment the SED model is configured to produce the SED Hardness data in response to the torque data, RPM data, force on bit (FOB) data and rate of penetration (ROP) data of the MWD data.

In an embodiment the SED model is configured to take into account characteristics of the drill bit.

In an embodiment the method includes generating the ground hardness predictions by applying the deep learning hardness prediction to a Mining Automation System (MAS) Hardness Model.

In an embodiment the method includes correcting and filtering the DL hardness prediction data, ROP hardness data and SED hardness data to thereby produce filtered hardness data and predictions.

In an embodiment the MAS Hardness model is responsive to the filtered hardness data and predictions to thereby produce the hole-by-hole ground hardness predictions.

In an embodiment the hole-by-hole ground hardness predictions include one or more hardness values determined to estimate a hardness profile down each hole.

In an embodiment the deep learning model is configured set ground hardness thresholds by referring to historical geological data.

In an embodiment the method includes providing the filtered hardness data and predictions to a ground type classifier wherein the ground type classifier is trained to generate ground classifications for the bench below from the filtered hardness data and predictions.

In an embodiment the method includes providing a pattern assist tool (PAT) to generate a bench below drill pattern (BBDP) based on at least the hole-by-hole ground hardness predictions.

In an embodiment the PAT is configured to produce the BBDP based on the hole-by-hole hardness predictions and data from one or more sensors of post-blast machines used to transport and process material blasted from the current bench.

In an embodiment the method includes providing a Materials Optimization Tool (MOT) that includes the one or more sensors of the post-blast machines.

In an embodiment the one or more sensors of the post-blast machines include one or more of a:
crusher power consumption sensor for sensing power consumption of a crusher;
particle size sensor for sensing size of particles leaving the crusher; and
throughput sensor for detecting the rate of flow of material at one or more points subsequent to the crusher.

In an embodiment the throughput sensor comprises:
a lump throughput sensor for detecting lump-size material at the one or more points subsequent to the crusher; and/or
a fines throughput sensor for detecting fines at the one or more points subsequent to the crusher.

In an embodiment the one or more sensors of the post-blast machines include machine sensors of dig units arranged to generate dig energy data.

In an embodiment the machine sensors of the dig units are arranged to generate dig rate data.

In an embodiment the machine sensors of the dig units include one or more cylinder sensors for sensing hydraulic pressure of one or more hydraulic actuation cylinders of the dig units.

In an embodiment the machine sensors include one or more of a cylinder pressure transducer, and a joint angle encoder.

In an embodiment the one or more hydraulic actuation cylinders include a boom retract cylinder, a stick extend cylinder and a bucket extend cylinder.

In an embodiment the PAT is configured to take into account local geotechnical surfaces data when producing the bench below drill pattern.

In an embodiment the method includes operating autonomous drilling systems (ADS) to execute a current bench drill pattern to perform the blast hole drilling where the current bench drill pattern is updated to the bench below drill pattern determined by the pattern assist tool (PAT) subsequent to blasting, digging and crushing of the current bench.

In an embodiment the method includes determining hole-by-hole charge specifications for holes in the current bench from the hole-by-hole ground hardness predictions.

In an embodiment the method includes providing a blast assist tool (BAT) which is responsive to the hole-by-hole ground hardness predictions to thereby produce the hole-by-hole charge specifications.

In an embodiment the BAT produces the hole-by-hole charge specifications by determining one or more of explosive density, explosive mass and stem height for each blast hole of the current bench.

According to another aspect there is provided a method for determining a drill pattern for a bench (“bench below drill pattern” (BBDP)) that is below a current bench, the method comprising:
generating measure while drilling (MWD) data from blast hole drilling of the current bench according to a current bench drill pattern (CBDP);
providing a hardness model that is responsive to the MWD data of the current bench to thereby produce hole-by-hole ground hardness predictions of the current bench; and
determining the BBDP by projecting the hole-by-hole ground hardness predictions of the current bench to the bench below; and
updating the CBDP to the BBDP prior to blast hole drilling the bench below.

In an embodiment the hardness model includes a deep learning model that is trained to generate deep learning hardness predictions (DL hardness predictions) from the MWD data.

In an embodiment the hardness model includes a fallback model that is configured to monitor torque data and air pressure data of the MWD and to produce a fallback detect signal therefrom indicating presence of drill hole fallback or malfunction of the pressurized air delivery arrangement.

In an embodiment the hardness model is responsive to the fallback model and is configured to discard one or more data making up the MWD data upon receiving the fallback detect signal.

In an embodiment the hardness model includes a rate of penetration (ROP) model that is responsive to ROP data of the MWD data, wherein the ROP model is configured to produce ROP hardness data indicating a hardness value at a position of the current bench.

In an embodiment the hardness model includes a specific energy of drilling (SED) model that is configured to estimate a SED value and generate SED hardness data based on the estimated SED value.

In an embodiment the hardness model includes a correct-and-filter assembly that receives the DL hardness predictions, ROP hardness data and SED hardness data and produces filtered hardness data and predictions therefrom.

In an embodiment the hardness model further includes a mining automation system (MAS) hardness model that is configured to produce the hole-by-hole ground hardness predictions in response to the filtered hardness data and predictions.

In an embodiment the method further includes providing the filtered hardness data and predictions to a ground type classifier

In an embodiment determining the BBDP is further based on local geotechnical surfaces data.

In an embodiment determining the BBDP is further based on data from material optimization tool sensors.

In an embodiment the data from the material optimization tool sensors includes one or more of:
particle size data indicating size of particles coming out of a crusher crushing blasted material of the current bench;
crusher power consumption data;
dig energy data, being data indicating energy required to dig the blasted material of the current bench;
dig rate data;
fines throughput data, being data indicating fines leaving a post-crusher material processing plant;
lump throughput data, being data indicating lumps leaving the post-crusher material processing plant.

In an embodiment the method includes determining hole-by-hole charge specifications based on the hole-by-hole ground hardness predictions.

In an embodiment the hole-by-hole ground hardness predictions are determined in the form an effective powder factor and a mass of explosive for each hole.

### BRIEF DESCRIPTION OF THE DRAWINGS

Preferred features, embodiments and variations may be discerned from the following Detailed Description which provides sufficient information for those skilled in the art to perform the various embodiments and aspects. The Detailed Description is not to be regarded as limiting the scope of the preceding Summary. The Detailed Description will make reference to a number of drawings as follows:

FIG. 1 depicts an Autonomous Drilling System (ADS) 1 shown drilling a blast hole in partial cross section and including sensors 2 which configured to generate Measure While Drilling (MWD) data.

FIG. 2 depicts a number of ADS in operation, each sending MWD data across a data network to a central processing assembly in the form of a specially programmed server.

FIG. 3 is a side view of an open cut mine wall showing a number of benches and indicating geotechnical surfaces being boundaries between material domains (sometimes called “geozones”) of differing hardness.

FIG. 4 is a top plan view of a bench prior to blasting showing a three different blast hole patterns for each of three geozones of different hardness.

FIG. 5 is a cross sectional view of a blast hole prior to detonation of explosion therein, showing the explosive and a stem of lump size material above the explosive.

FIG. 5A is a cross sectional view of a blast hole prior to detonation of explosion therein wherein due to a varying hardness profile of the material in which the hole is formed, density of explosive down the hole is varied from a low density beneath the stem, toward the top of the hole, to high density at the bottom of the hole.

FIG. 6 is a stylized arial view of an open cut mine subsequent to blasting of a bench wherein dig units, such as loaders, load haul trucks with blasted material and wherein the loaded haul trucks transport the blasted material for subsequent processing such blending of material in stockpiles, crushing, rail load-out and rail transportation to a port.

FIG. 7 is a block diagram of a vehicle control system of vehicles depicted in FIG. 6 such as the haul trucks and loaders.

FIG. 8 is a flowchart depicting a method for generating blast hole patterns and charge design specifications for blast holes of the blast hole patterns to produce blasting that is neither over-blasted nor under-blasted in order to optimize material properties, such as lump dimensions of blasted material.

FIG. 9 is a detail of the hardness modeling box of the flowchart of FIG. 8.

FIG. 9A is a graph depicting variation of torque, and of air pressure, related to hole depth, for purposes of explaining the effect of fallback of material into a hole.

FIG. 10 is a graph relating the depth of a drill bit to time taken to reach that depth and showing bogging events in which the drill has been retracted.

FIG. 11 is a graph depicting training and validation loss during training of Deep Learning Model 89 (FIG. 9).

FIG. 12 depicts a number of histograms each showing a material type hardness distribution per hole for each of seven autonomous drills A to G.

FIG. 13 is a detail of the Material Optimization Tool (MOT) box of FIG. 8.

FIG. 14 is a side view of an excavator, identifying various cylinders which are monitored for determining Specific Dig Energy (SDE).

FIG. 15 is a graph illustrating the amount of power expended by a particular digger (i.e., a loader) over a dig cycle compared to its dig rate.

FIG. 16 is a graph illustrating that there is “jerkiness” in a cylinder pressure signal when digging hard ground.

FIG. 17 is a graph showing a “smooth” pressure signal from the cylinder when digging soft ground.

### DETAILED DESCRIPTION OF PREFERRED EMBODIMENTS

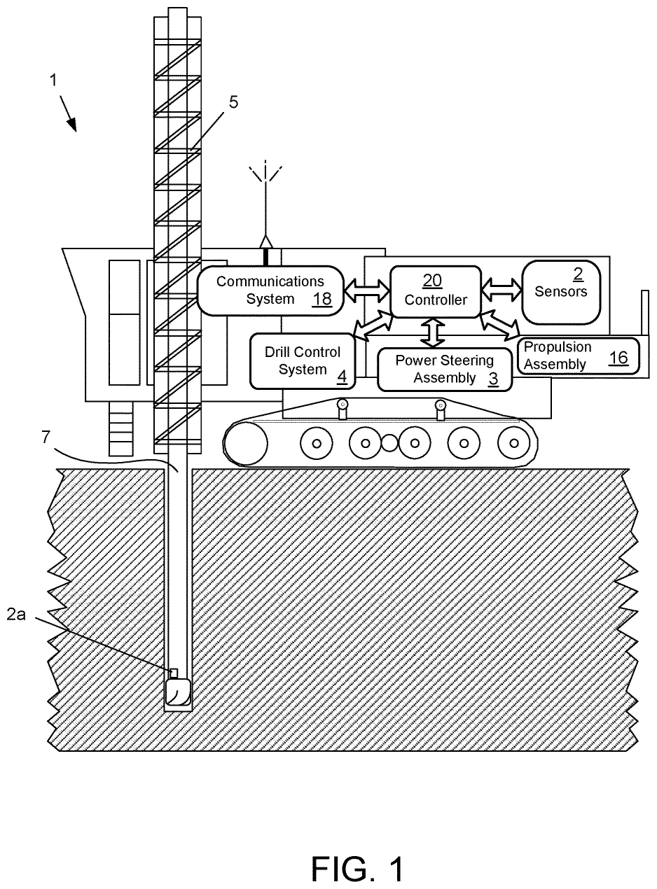

FIG. 1 depicts an Autonomous Drilling System 1 (ADS), which comprises an autonomous vehicle that has a remotely controllable power steering assembly 3 and a drill support frame 5 from which extends a drill 7. Autonomous Drilling Systems such as ADS 1 enable a remote operator using a single console at a distant location to control numerous ADS simultaneously, improving precision and equipment utilization. ADS 1 is equipped with a number of sensors 2 including Measure While Drilling (MWD) sensors that collect MWD data. In a preferred embodiment each ADS is fitted with sensors to obtain MWD data that can be processed to reveal properties of the ground being drilled. In an embodiment the ADS 1 includes an air pressure sensor 2a which measures the air pressure from a pressurized air delivery arrangement, such as a nozzle, located near the drill bit and located down the hole in use. The pressurized air delivery arrangement is used to apply pressurized air to blow cuttings from the drill hole. The sensors 2, 2a are coupled to a controller 20 of the ADS 1 which receives and sends commands and data from the sensors to a remote site via onboard radio communications system 18. Controller 20 is configured to control and monitor drill control system 4, power steering assembly 3 and propulsion assembly 16. Typical MWD sensors used by ADS 1 are set out in the following Table 1.

TABLE 1
Measure While Drilling Sensors

| | Symbol | Units | Parameter |
|---|---|---|---|
| 1 | T | Nm | Torque |
| 2 | ROP | m/s | Rate of Penetration |
| 3 | RPM | Revs/min | Rotation speed or rate of rotation |
| 4 | FOB | N | Force on Bit |
| 5 | AP | kPa | Air Pressure |

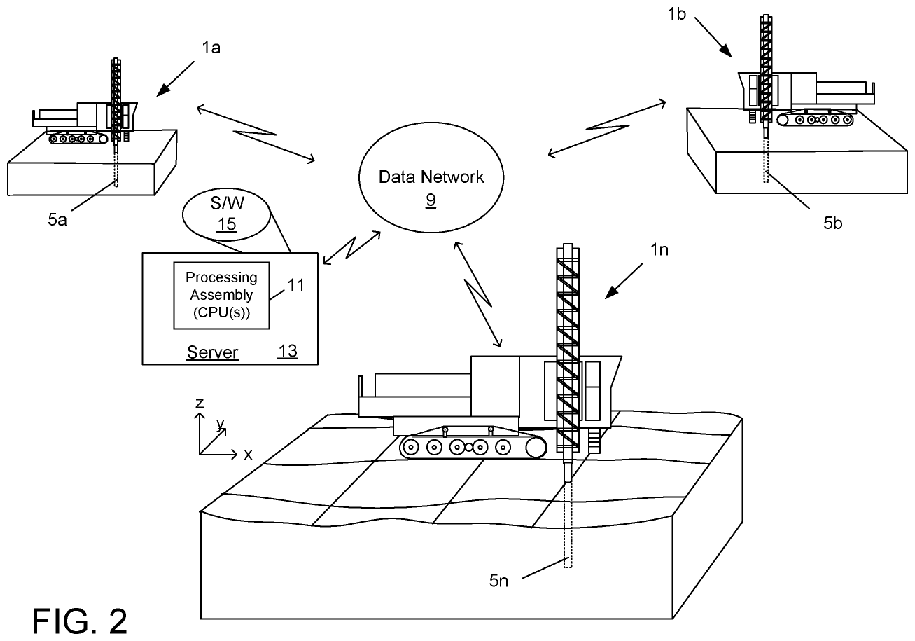

As illustrated in FIG. 2, the MWD data that is set out in Table 1 is transmitted via data network 9 to a processing assembly in the form of server 13, specially configured by software 15 to implement a number of models that process the MWD data as will be explained. It will be realized that the server 13 may be implemented as a distributed system, for example it may be a virtual server that is cloud hosted.

The drill control system 4 (FIG. 1) of each ADS 1a, . . . , 1n is arranged to modulate force on bit (FOB) to target a pre-specified rotation torque. If the torque is high, the drill control system 4 lowers the FOB. If too low, it raises the FOB. While this is quite similar to how an experienced operator may control the drill 7, the drill control system 4 is more precise. This means that when a drill hits a transitional ground, i.e., from soft to medium, the instantaneous impact of this transition to torque, rotation speed and FOB is amplified. This is because the drill control system inputs are under the strict control of an automated algorithm and hence the impact of control inputs to ROP is minimised.

In manual drilling, different operators can have more influence on ROP due to experience levels, tiredness, and operational pressures for example increase of FOB to drill faster. It has been found that if the drilling inputs are not precisely controlled the result is a significant error to predictions.

Secondly, an automated drill mandates industrial quality, reliable and maintainable, working sensors to provide the feedback loop required for automation. The control system in an automated drill expects a certain output given an input and if an output signal is not within the expected range, the system can either stop drilling or raise alarms. This provides significant help with ensuring that sensors are maintained.

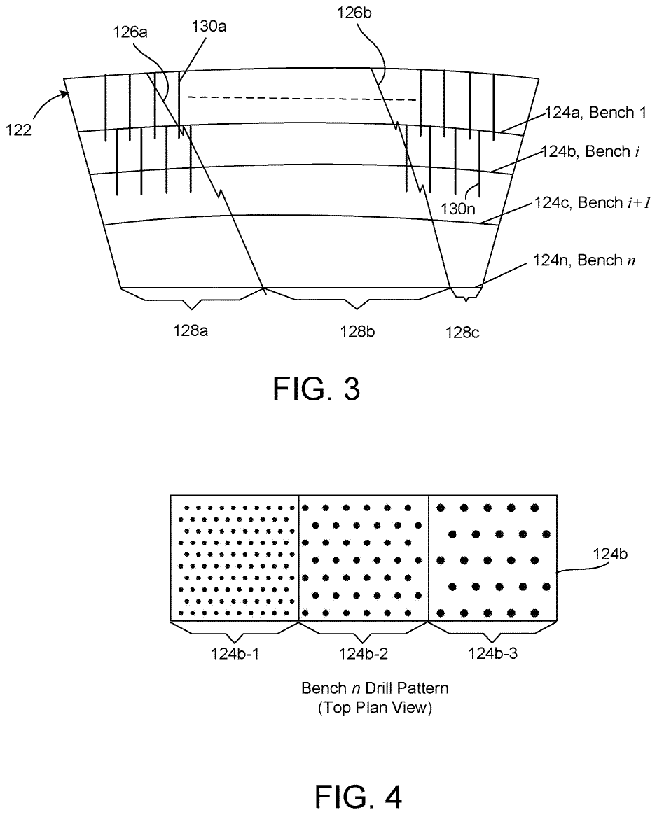

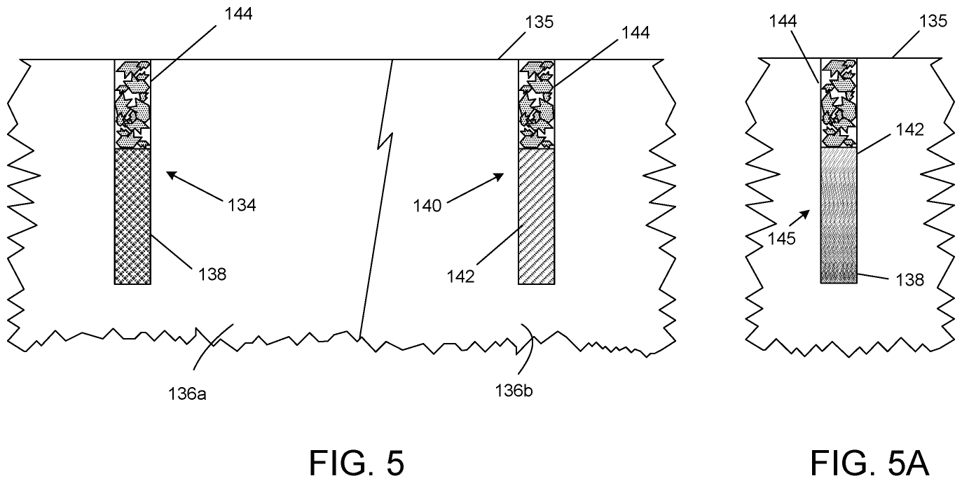

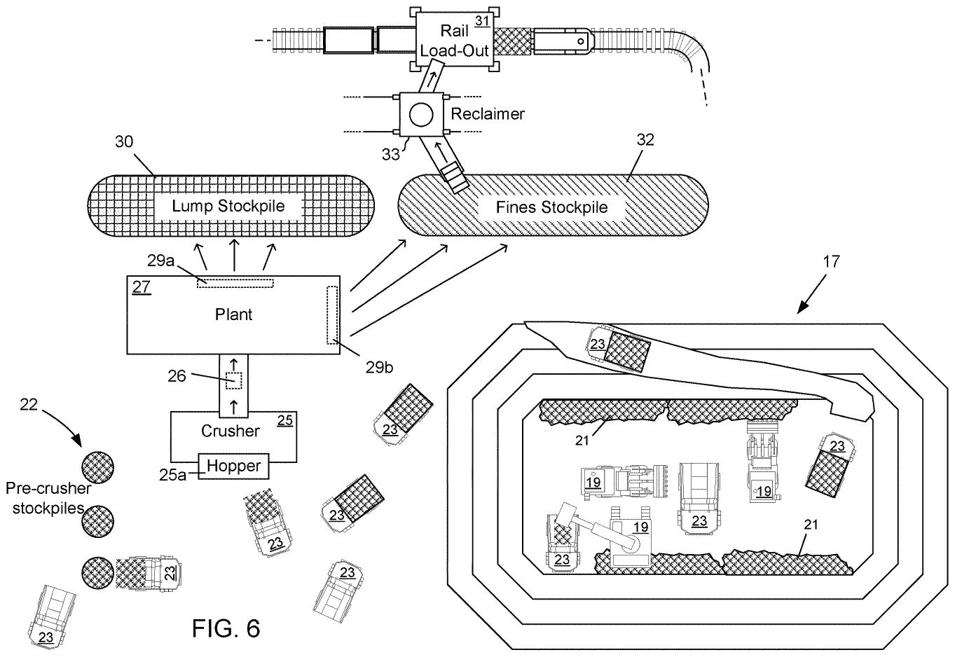

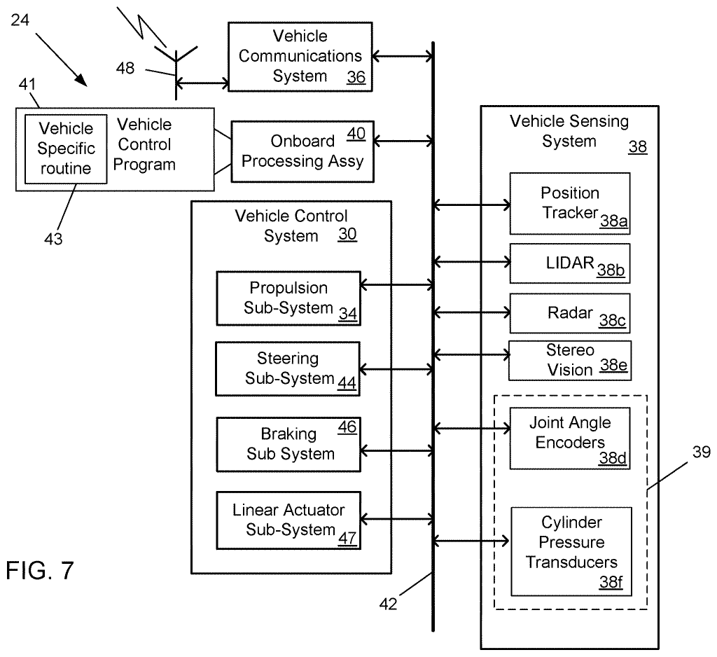

FIG. 3 is a side view of an open cut mine wall 122 showing a number of benches 124a, . . . , 124n and indicating geotechnical surfaces 126a, 126b being boundaries between material domains (sometimes called “geozones”) of differing hardness such as hard geozone 128a, medium hardness geozone 128b and soft geozone 128c. The geotechnical surfaces 126a, 126b are typically ascertained during initial surveys of the mining site using exploratory drilling, which is undertaken at horizontally sparser locations than MWD blast mining. For example, the exploratory drilling is typically done on a grid with a 50 m×50 m spacing whereas blast hole drilling such as that done by the ADS 1 to make blastholes 130a, . . . , 130n is done on a finer grid for example of the order of 6 m×7 m spacing. However, exploratory drilling is done to a much greater depth than blast hole drilling in order to be able to ascertain vertical dimensions of geological domains. As will be discussed, MWD data obtained from ADS drilling rigs whilst drilling blast holes for bench i (e.g. bench 124a) can be processed taking into account the geozones, and along with data from loading vehicles and from ore processing plant such as crushers and blending stations, to generate blast patterns and charge specifications for blast holes for the next bench down, i.e., bench i+1 (i.e. bench 124b) in the FIG. 3, to be blasted. For example, FIG. 4, is a plan view of a blast pattern for bench 124b where it will be observed that tighter burden and spacing for the blast hole pattern has been produced for the hardest geozone 124b-1, whereas a large burden and spacing blast hole pattern has been produced for the soft geozone 124b-3 and an intermediate burden and spacing blast hole pattern for the medium hardness geozone 124b-2 between them. In addition to producing a blast pattern taking into account the MWD and other data, embodiments provide a Blast Assist Tool (BAT, item 77a of FIG. 7, to be discussed) that is configured to process hardness data based on the MWD information and thereby produce charge specifications for each blast hole in the blast hole pattern. The charge specifications include the mass and density of the explosive to be used taking into account factors such as ground hardness, as illustrated in FIG. 5. In FIG. 5 a blast hole 134 drilled below ground surface 135 into a hard geozone 136a is shown charged with a high density (high energy) explosive 138, whereas a blast hole 140 drilled into a relatively soft geozone 136b is shown charged with a low density (low energy) explosive 142. Stemming 144 covers both explosive charges 138, 142. Where MWD data is captured it is typically obtained at regular intervals such as every 0.1 m down as blast holes such as 134, 140, are being drilled. Accordingly, the BAT 77a determines charge specifications that vary between blast holes taking into account data, for example the data may include hardness data that has been previously collected for earlier benches. With reference to FIG. 5A, the charge specifications may vary vertically down a blast hole 145 so that, for example, denser explosive 138 is used further down the hole and less dense explosive 142 is used at the top of the blast hole. The variation vertically in the determined charge specifications is possible because the hole-by-hole ground hardness predictions may include one or more hardness values determined to estimate a hardness profile down each hole. FIG. 6 is a stylized arial view of an open cut mine 17 subsequent to blasting of a bench, in which loading vehicles 19 load blasted material 21 into haul trucks 23. Loaded haul trucks 23 transport the blasted material to pre-crusher stockpiles 22 for subsequent loading into hopper 25a of crusher 25. The crusher 25 crushes the blasted material that it receives. Material exiting the crusher is then blended at a blending plant 27. The crushed and blended ore is then temporarily stored in lump and fines stockpiles 30, 32 before transferred by reclaimer 33 to Rail Load-Out station 31 for rail transportation to a port or other dispatch center. The loaders 19 and haul trucks 23 are fitted with various sensors and actuators as indicated in FIG. 7. Vehicle control and sensing system 24 includes a vehicle sensing system 38 which in turn includes a number of assemblies 38a-38f for determining the vehicle's pose, which will typically comprise data such as the vehicle's position, speed, direction, proximity to other vehicles orientation and joint angles. Position tracker 38a may comprise a Global Positioning System (GPS) receiver which is configured to generate information about at least the position of the vehicle at each of a series of times, for example five second intervals. The position tracker may also be configured to triangulate a position estimate from terrestrial transmitters such as wireless transceivers, which are part of data network 9. The position tracker 32 may also include gyroscopes, accelerometers and/or other apparatus that can also be used to generate position signals indicating the location of the vehicle to which it is fitted within the mine environment. The position tracker 38a is able to ascertain at least the vehicle's position at progressive times as it travels through the area. Vehicle tracking system 38 also includes LIDAR sensor 38b, radar sensor 38c and stereo vision sensors 38e for estimating proximity to obstacles and for assisting in collision avoidance and loading and unloading of material. Joint angle encoders 38d are also provided in the vehicle tracking system to generate signals indicating the angle of various joints including steering angle, bucket angle, and loader arm angles.

Where the vehicle is a loader then the various sensors include Dig Energy sensors 39 which include the joint angle sensors 38d and cylinder pressure transducers 38f that provide an indication of how much energy is needed to load the blasted material 21 into the haul trucks 23.

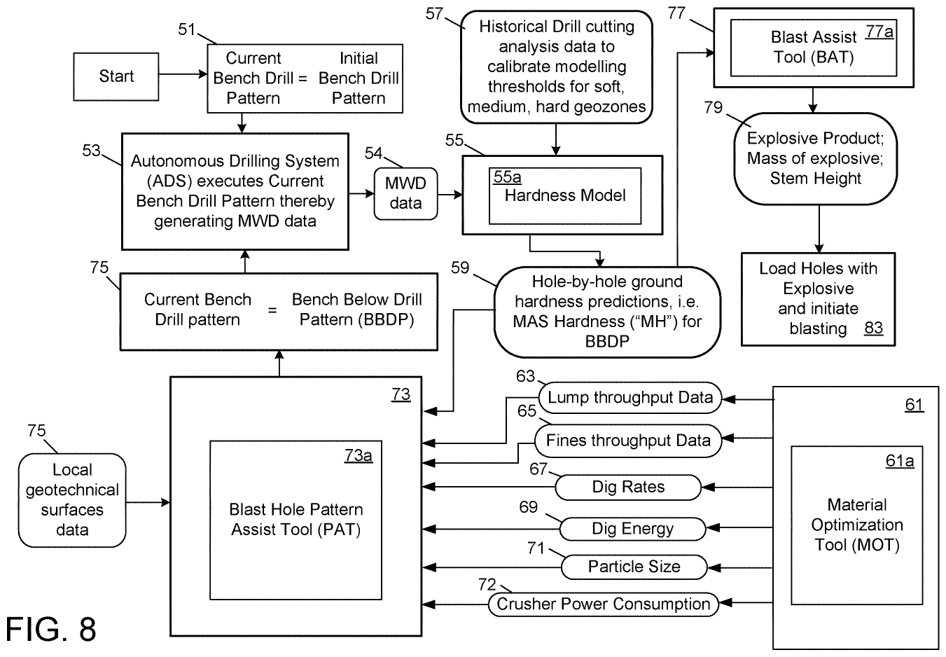

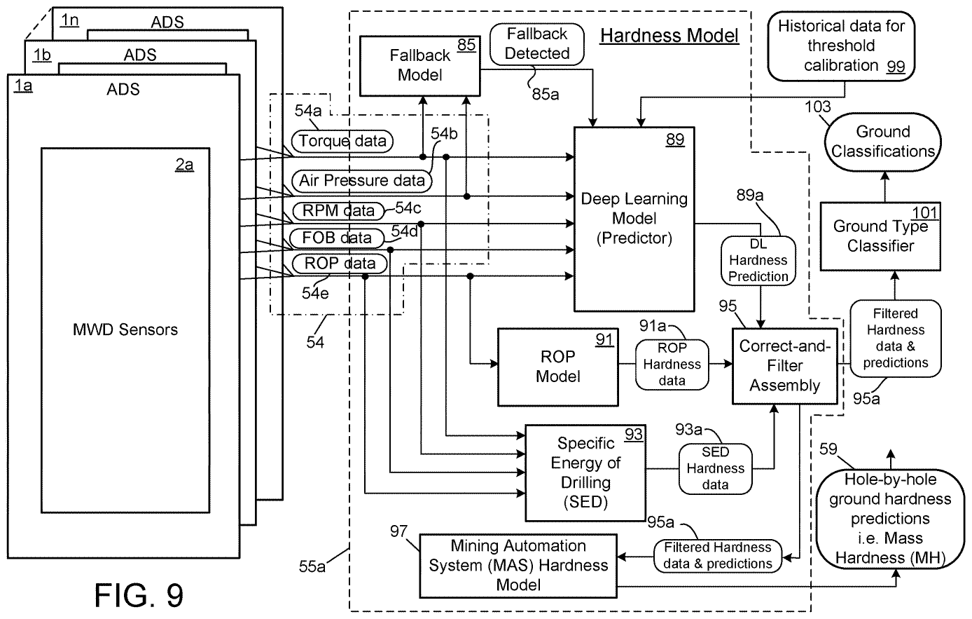

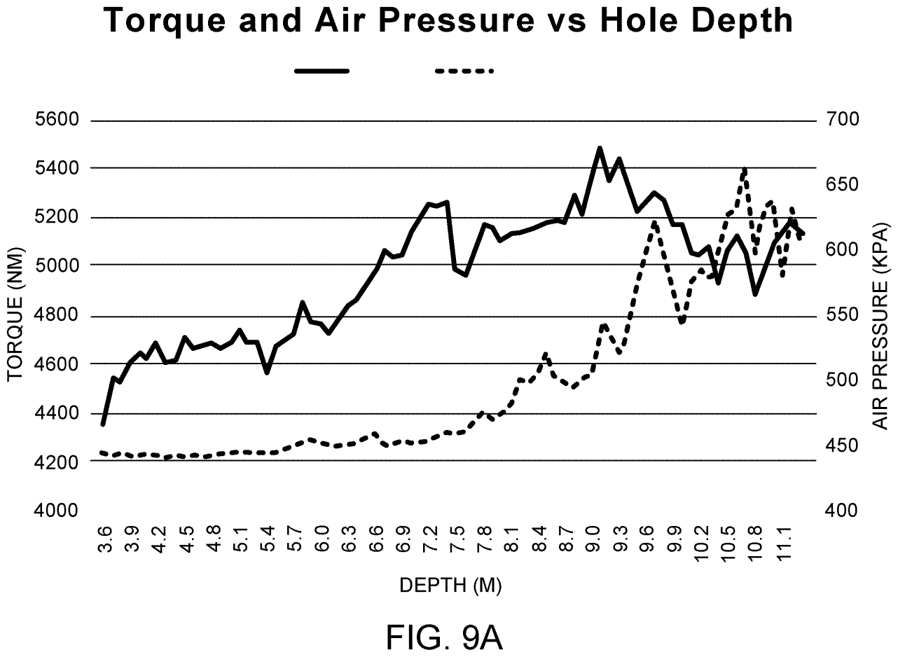

Turning now to FIG. 8, a flowchart of a method, that is implemented by server 13 as configured by software 15, to cause improved blasting in the mine 17 will now be described. Initially at box 51, a current bench drill pattern is initialized and loaded into ADS 1a (FIG. 2). A drill pattern specifies the burden and blast hole spacing and where each hole in the pattern should be. Similarly, bench drill patterns for additional ADS 1b, . . . , 1n (FIG. 2) are also provided with initialized bench drill patterns. However, the present exemplary description of the preferred method will be given primarily in relation to ADS 1a. At box 53 the server 13 instructs ADS 1a to execute the current bench drill pattern so that the ADS 1a generates the MWD data 54, as set out in Table 1. The server 13 receives the MWD data 54 via data network 9 and applies them to a Hardness Model 55a at box 55, as will subsequently be described in more detail with reference to FIG. 9. The Hardness Model 55a processes the MWD 54 with reference to thresholds that it uses to predict if the data is associated with particular geological properties, such as soft, medium or hard ground types. The thresholds are calibrated using historical data 57 which geological studies have previously obtained and which associate MWD data with the different types of geological characteristics.

Hardness Model 55a generates Mass Hardness (MH) predictions 59, for ground hardness of a bench-below drill pattern. At box 73 the MH predictions 59, along with additional data values 63, . . . , 72 from Material Optimization Tool (MOT) 61a are applied, at box 73, to a blast hole Pattern Assist Tool (PAT) 73a. The PAT 73a also receives local geotechnical surfaces data 75 for the region in which the MWD has been gathered. The PAT is configured to produce the BBDP based on the hole-by-hole hardness predictions and one or more sensors of post-blast machines used to transport and process material blasted from the current bench.

The data values 63, . . . , 72 include one or more sensors of post-blast machines. For example the sensors may include a lump throughput sensor for detecting lump-size material from the post-crusher material processing plant, a fines throughput sensor for detecting fines from the post-crusher material processing plant, a crusher power consumption data from a power sensor of the crusher and a particle size sensor for sensing size of particles leaving the crusher.

PAT 73a processes the various inputs 59, 63, . . . , 72, 75 to produce the Bench Below Drill Pattern (BBDP), which is a drilling pattern for the next bench down, e.g. Bench i+1 in FIG. 3. At box 75 the current bench drill pattern is updated to the Bench Below Drill Pattern, prior to the method proceeding again to box 53. PAT 73a creates the BBDP from hole-by-hole ground hardness predictions 59 from Hardness Model 55a, local geotechnical surfaces data 75 and data from Material Optimization Tool (MOT) 61a.

The hole-by-hole ground hardness predictions may include one or more hardness values determined to estimate a hardness profile down each hole.

The data that PAT 73a receives from MOT 61a includes: Lump Throughput Data 63, which is data from plant 27 describing the volume of lumps above a certain size exiting the plant 29. Lumps are fragments that fall within a predetermined desirable range of dimensions. Too few lumps, will cause a loss of the “lump premium” and thus a decrease in the price per ton of material. Fines Throughput Data 65, which is data indicating the volume of material exiting the plant 29 that comprises fines. Too large a volume of fines decreases the price per ton of material and thus is undesirable. Dig Rates data 67, which is data from the loaders 19 that indicates the rate at which the loaders are able to dig the blasted material 21.

A high dig rate may be associated with the blasted material being over-blasted whereas a low dig rate may indicate the presence of very large, blasted material fragments and thus be associated with under-blasting. Dig Energy data 69, which is data from the Dig Energy sensors 39 of the loaders 19 and indicates how much energy is being used to load the blasted material into the haul trucks.

Particle Size data 71, which is data from a particle size sensor 26, at the exit of crusher on the primary crusher belt, indicates the size of the crushed material exiting the crusher.

Crusher power consumption data 72, which is data from the crusher's electrical system indicating the amount of electrical power that the crusher consumes to generate the crushed material that exits the crusher, with particle sizes sensed by the particle size sensor 26. PAT 73a processes the crusher power data together with the particle size sensor data to improve the accuracy of the BBDP that it generates. Exemplary Pseudo code for the PAT 73a is as follows:
Extend BBDP dimension by 20 m in all directions
Get all holes above BBDP
Get geotechnical surfaces above BBDP
Create 3D Geozones where bottom of Geozone starts at BBDP and top of Geo-zone ends at the geotechnical surface
Divide BBDP into 5 m×5 m grid
For each grid
{
Get all holes within geozone enclosing the grid
Using Inverse Distance Square, project MAS Hardness Raw, average SED, average ROP values for each of the holes within Geozone to the centroid of the grid so that each grid contains a value of MAS Hardness Raw, SED and ROP
IF (MAS Hardness Detection=TRUE) [see Pseudo code above]
{
Store
MAS Hardness Raw, SED and ROP for that grid in the database
}
For BBDP
{
Using Alphashape and Geopandas libraries and the MAS Hardness Raw field, cluster Soft, Medium, Hard and Extra Hard grid centroids and create Soft, Medium, Hard and Extra Hard polygons (the polygons may overlap each other)
To calculate MAS Hardness execute below in the given order
IF Grid Centroid within Extra Hard Polygon, THEN MAS Hardness=Extra Hard
ELSE IF Grid Centroid within Hard Polygon, THEN MAS Hardness=Hard
ELSE IF Grid Centroid within Soft Polygon, THEN MAS Hardness=Soft
ELSE IF Grid Centroid withing Medium Polygon, THEN MAS Hardness=Medium
Store MAS Hardness for each grid in the database (lookup tables are later used to specify burden, spacing and diameter for holes that are in a specific cluster of grids using MAS Hardness as input to the lookup table)
}

The hole-by-hole ground hardness predictions from Hardness Model 55a are also passed to box 77 where it is processed by a Blast Assist Tool (BAT) 77a. Blast Assist Tool 77a is configured to process the Mass Hardness information 59 for the current bench and determine charge specifications 79 for the blast holes prior to loading the holes in the current bench with explosive at box 83 and then blasting the current bench. In the presently described embodiment the charge specification includes the type of explosive product to be used (different products have different densities and varying explosive force), mass of that explosive product to use and stem height for the particular hole.

Example pseudo code for operation of the BAT 77a is as follows:
Extract MWD data for a Current Bench
Organise MWD data so that it is grouped for each hole and in increasing depth and by drill type
Filter out the top 3 m of MWD data due to damage from blast above
For each hole in pattern
{
Apply MWD data into the Drill Type specific DL model and get Probability of Hardness at 0.1 m depth increments
Get SED data at 0.1 m depth increments (no processing required-SED data is extracted from Mine Automation System as part of the MWD dataset)
Get ROP data at 0.1 m depth increments (no processing required-ROP data is extracted from Mine Automation System as part of the MWD dataset)
Apply Fallback filter (see Pseudo code above)
Apply Correct-and-Filter (see Pseudo code above)
Break DL, SED and ROP datasets to top and bottom half of the hole (if hole is 13 m, top half is from 3 m to 8 m and bottom half is 8 m to 13 m due to omission of the top 3 m)
Using the thresholds outlined in Table 3, classify top and bottom half of hole to into Soft, Medium and Hard categories for each of DL, SED and ROP
If (Fallback=FALSE) AND (MAS Hardness Detection=TRUE) [see Pseudo codes above]
{
MAS Hardness Raw=relative hardness for the hole calculated using schema in Table 2 and converted to integer representation for each hardness category
Store MAS Hardness Raw, average SED for the hole, average ROP for the hole and top and bottom DL hardness categories for the hole in the database (to be used by PAT later)
}
}
For Current Bench
{
Using Alphashape and Geopandas libraries and the MAS Hardness Raw field, cluster Soft, Medium, Hard and Extra Hard holes and create Soft, Medium, Hard and Extra Hard polygons (the polygons may overlap each other)
To calculate MAS Hardness execute below in the given order
IF Hole within Extra Hard Polygon, THEN MAS Hardness=Extra Hard
ELSE IF Hole within Hard Polygon, THEN MAS Hardness=Hard
ELSE IF Hole within Soft Polygon, THEN MAS Hardness=Soft
ELSE IF Hole withing Medium Polygon, THEN MAS Hardness=Medium
Store MAS Hardness for each hole in the database (used to specify explosive mass and explosive product for each hole)

FIG. 9 shows a detail of the hardness model 55a of the flowchart of FIG. 8. As previously discussed, each ADS 1a, . . . , 1n generates MWD data 54, which in the present example comprises torque data 54a, air pressure data 54b, RPM data 54c, force on bit (FOB) data 54d and Rate of Penetration (ROP) data 54e.

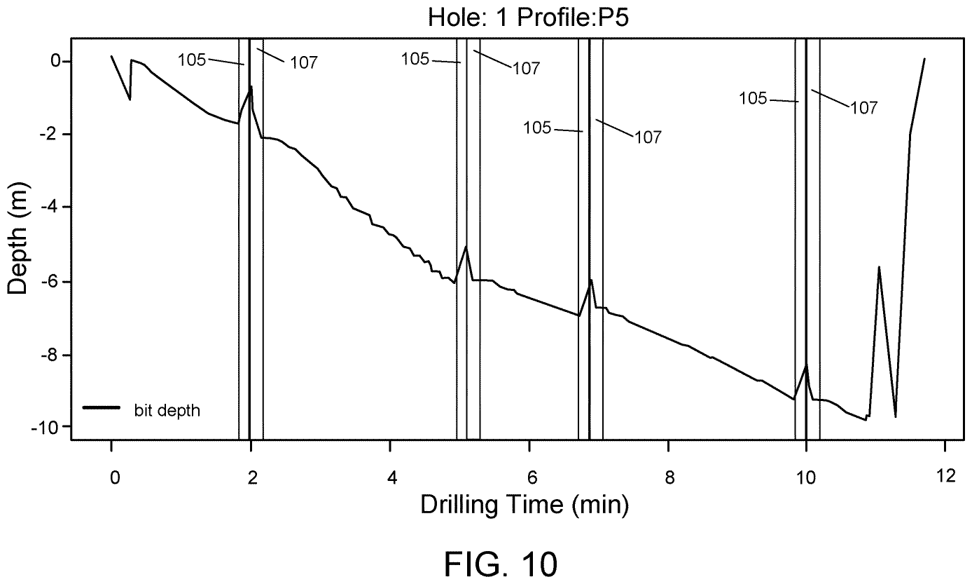

The MWD data is filtered to only include the first pass of drilling, i.e. if there is a bog event where the bit was retracted, the process parameters are only stored for the initial drill, not retract and re-drill. For example, in FIG. 10, sections 105, 107 of depth data are not analysed.

Fallback Model 85 monitors the Torque data 54a and the Air Pressure data from the MWD sensors 2a. Fallback is a physical phenomenon that may occur during drilling in which the sides of the hole being drilled collapse and fall into the hole. Air pressure from a pressurized air delivery arrangement, such as a nozzle, is correlated to the amount of material present around the drill bit. The more material, the more pressure required to blow the material out of the hole. When there is a significant amount of fallback and if there is not enough pressure to blow the material, it will lead to a bogging event as illustrated in FIG. 10. Fallback from sides of the holes is the primary reason for increased presence of material around the drill bit. Another reason may be that the air nozzle is faulty and unable to blow out the drill cuttings efficiently even when there is no fallback. Both of the conditions lead to high air nozzle pressure and high torque (and incorrect classification of hardness).

When fallback or drill cuttings accumulate at the bottom of the hole then the Inventors have observed that they increase the torque on the drill which the Drill Control System 4 reacts to by decreasing Force On Bit to prevent damage to the drill and drill bit and avoid high levels of vibration. Consequently, the Rate of Penetration drops, which to an observer would appear to indicate that the drill is encountering unusually hard ground whereas in fact fallback is typically associated with soft ground. The Fallback Model 85 is configured to produce a Fallback Detected signal 85a if the Air Pressure data 54b and the Torque data 54a indicate that either fallback or a malfunction of the pressurized air delivery arrangement, e.g. the nozzle, has occurred. For example, if the air pressure data indicates an air pressure above a predetermined air pressure threshold, and the Torque data is also above a predetermined torque threshold value, then the Fallback Model 85 produces the Fallback Detected signal 85a. Additionally air-pressure and torque may be tracked as a function of hole depth. There is a greater chance of fallback with increase in depth. So the model normalises air pressure and torque at bottom and top halves of the hole to minimize false alerts. By normalize, it is meant that the model averages the torque and air pressure for the top and bottom halves. These averages are then used as part of the thresholds. Thresholds for each are determined by analysing holes that produce hard ground predictions despite ground being known to be soft.

In one embodiment the Fallback Model is prepared as follows:
1. Identify >1000 of holes where hardness is incorrectly classified as hard. This misclassification can either be determined by selecting holes that are classified as hard in obvious soft zones (use of geology) or where two drills operate side by side but provide opposite hardness classification. In the case of latter, sign of malfunction of the pressurized air delivery system, the hardness zones follow the distinct boundaries of holes drilled by two different drills
2. Identify >1000 holes where hardness is correctly classified as hard. Using similar approach to first point and where two different drills operate side by side and provide similar hardness
3. Establish the following means for misclassified holes. Top 3 m of the hole is discarded due to damage from blast above and the remaining depth is divided in equal halves to top and bottom of hole:
   a. mean across holes (mean bottom half (Air Pressure)/mean top half (Air Pressure))
   b. mean across holes (mean bottom half (Torque)/mean top half (Torque))
   c. mean across holes (max bottom half (Torque)/mean top half (Torque))
   d. mean across holes (mean (ROP))
   e. mean across holes (max bottom half (Rate of Change in Air Pressure))
   f. mean across holes (max bottom half (Rate of Change in Air Torque))
4. Repeat item 3 for correctly classified holes
5. Adjust thresholds between 3a (misclassified) and 4a (correctly classified) and 3b and 4b . . . , etc for fields in a to f until set % (i.e. >70%) of Fallback holes can be identified correctly and set % (i.e. <10%) of good holes are falsely identified as Fallback. These are the thresholds used in the below pseudo code.

FIG. 9A is a graph of typical Air Pressure and Torque across depth for a hole with Fallback.

Exemplary pseudo code by which the Fallback Model detects the presence of fallback is as follows:
Set Fallback to TRUE IF
{
(bottomAirAv/topAirAv)>Threshold((bottomAirAv/topAirAv)) AND
(bottomTorqueAv/topTorqueAv)>Threshold(bottomTorqueAv/topTorqueAv)
AND
(bottomAirMax/topAirAv)>Threshold(bottomAirMax/topAirAv) AND
(bottomTorqueMax/topTorqueAv)>Threshold(bottomTorqueMax topTorqueAv) AND
ropAv<Threshold(ropAv) AND
RateOfChangebottomAirMax>Threshold(RateOfChangebottomAirMax) AND
RateofChangeTorqueMax>Threshold(RateofChangeTorqueMax)
}

The Deep Learning Model 89 is responsive to the Fallback Detected signal 85a and thus is able to detect when some or all of the MWD data 54 should be ignored, so that the accuracy of the DL Hardness Prediction 89a that it produces is not adversely affected by MWD collected during either fallback or malfunction of the pressurized air delivery arrangement, e.g. a nozzle.

The Deep Learning Model 89 is a Long Short Term Memory model, which is a type of recurrent neural network (RNN) that is known in the field of machine learning. The Deep Learning Model 89 receives the MWD data 54 and the derived Fallback Detected data 85a. The Deep Learning Model has been trained with supervised learning, using large sets of MWD training data to produce a model that is able to predict ground hardness of the bench below from the MWD data 54 of the current bench. In a preferred embodiment the model consists of the following layers:
1. Masking—essentially filters out empty fields
2. First LSTM layer
3. Second LSTM layer
4. Time distributed layer

Supervised learning is used for creating the Deep Learning Model 89. Supervised learning consists of learning to map input data (in this case the MWD data) to known targets (density readings from down the hole). The particular model that is used is Long Short-Term Memory (LSTM). This model is particularly useful at analysing timeseries data to predicting near future outcomes. However, in hardness modelling, depth, instead of timeseries is used. For each 0.1 m increment depth of MWD data, the weighting of each MWD parameter is modified and compared to the density provided by the downhole tool (also at 0.1 m increment). This process is repeated 600 times (epochs) for all depth increments until there is minimal difference (loss) between actual and predicted measurements.

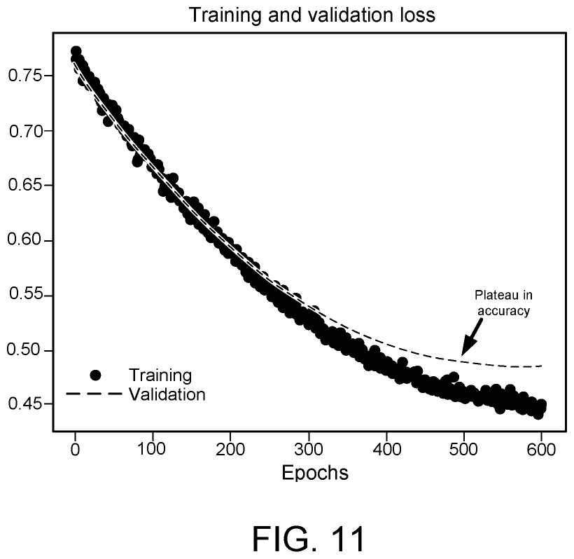

Approximately 30% of downhole data is reserved for validation. This is data that the model has not yet seen. It is used as a sense check to ensure weights adjusted by the training dataset are also applicable and accurate for the validation dataset. In FIG. 11, it can be observed that at around ~500 repeats, the validation loss (dotted line) plateaus at 0.49 and further training does not improve validation accuracy. This 0.49 validation loss corresponds to binary accuracy of 75.28% (see further below for definition of binary accuracy). Note that 0.45 training loss corresponds to 80.32% binary accuracy.

In order to describe the meaning of accuracy, the model data preparation needs to be understood. A dual density probe was used to collect the reference density target data. This probe is designed for exploration holes where the diameter of the holes is smaller, and rugosity of the holes are less/better (smoother hole walls).

In order to collect accurate density readings, the density probe needs to be flush against the hole with no air gap. These sections are detected using calliper readings integrated into the probe and often correspond to compensation factors of smaller than 0.2 g/cc (downhole service provider uses this compensation factor to convert the gamma readings to density-smaller compensation corresponds to areas where probe is flush against the wall). Further to compensation filtering, each hole data is visually inspected. Some of the holes (~10%) contained wildly different density readings per 0.1 m increment across the hole. These holes were omitted in entirety from model inputs. Further, any depth increments from good holes with compensation factor greater than 0.2 g/cc are omitted. The model ignores these small gaps in data.

For the next step in the process, each 0.1 m depth was classified into “hard” or “soft” categories. If density from the probe reading is greater than predetermined density reading, it was classified as hard and target depth was labelled “1”. The purpose of this classification is to create an approximation to hardness using density and to avoid training the model to predict absolute density. This would increase complexity of the model, but also the primary purpose was to predict hardness, not density.

The modelling uses material types where there is a linear relationship between hardness and density. Hence the model uses density as a proxy to predict the hardness. For each depth, the model outputs probability of ‘hard’, p(hard). When label data is 1 (hard) and predicted output p(hard) is 1, there is no loss for that depth. When label data is 0 (soft) and p(hard) is 1, there is maximum loss for that depth. While the model predicts binary hardness, soft or hard, p(hard) provides a prediction of hardness across the range, for example p(hard) of 0.5 could be considered medium hardness. More detail on hardness threshold calculations is provided later in this specification.

The model accuracy of ~75% is the percentage of depths where the density tool provides a reading above the ‘hard’ threshold and the model predicts ‘hard’ correctly. This accuracy is improved by converting each 0.1 m increment predictions for top and bottom half of hole. The primary reason for segregating top and bottom is that operations have capability to deliver different explosive energy to top and bottom of the charge column. Discussion of creation of a tertiary hardness (soft, medium, hard) is provided later in this specification.

As shown in FIG. 9, ROP Model 91 monitors the MWD ROP data and, based on how quickly the drill is progressing into the ground, generates an estimate of ground hardness of the current bench. The estimate is produced based on previously compiled correlations of ROP with ground hardness. The DL, SED and ROP data are ultimately used in both predictions of hardness for the current bench and predictions of hardness for the bench below.

High and low ROP zones represent the speed at which a drill penetrates rock and, in the past, may be used as a proxy for hardness. There are examples of cases where ROP aligns with shale bands from a structural model very well. Notwithstanding this, analysis of ROP performance and thresholding to identify soft, medium and rock using ROP is discussed in below along with how the standard ROP is converted into the ROP model (soft, medium, hard)

Specific Energy of Drilling Model 93 processes the Toque data 54a, RPM data 54c, FOB data 54d and ROP data 54e from the ADS MWD sensors 2a, to determine a Specific Energy of Drilling (SED) estimate, which it then uses to generate SED Hardness data 93a. SED is the amount of energy that must be used to destroy and remove a unit volume of rock from beneath the drill bit. Accordingly, there is a correlation between the value of SED that is required for drilling and the hardness of the ground being drilled. The SED Model 93 generates SED Hardness data 93a for the current bench being drilled.

Teale published concept of SED in 1965 where it is defined as the work required to drill through a unit volume of rock. It is calculated using the following formula:

$$e = Thrust compnonent + Rotation component = \frac{F}{A} + \frac{2 π NT}{Au}$$

$$where$$

$$e = Specific Energy of Drilling ( J / {m}^{3} )$$

$$F = thrust ( N )$$

$$A = cross sectional hole area ( {m}^{2} )$$

$$N = rotation rate ( Hz )$$

$$T = torque ( Nm )$$

$$u = penetration rate ( m / s )$$

The advantages of using SED for analysis of hardness is that is it takes rate of penetration, weight on bit, rotation speed and Torque. Reducing these to a single value is physically meaningful and it is correlated in the literature to energy required to break rock in other mechanisms such as SAG mills. From a theoretical point of view, it is less sensitive to changes in drilling configurations and bit age and is believed to reflect the properties of the rock better.

Correct-and-Filter Assembly 95 receives the DL hardness prediction 89a from Deep Learning Model 89, ROP Hardness data 91a from ROP Model 91 and SED Hardness data 93a from SED Model 93. The Correct-and-Filter Assembly 95 is configured to detect and filter out received data that is known, from previous records, to be inconsistent, anomalous or misleading. The Correct-and-Filter Assembly 95 stores filtered hardness data for access by the Mining Automation System (MAS) Hardness Model 97. Correct-and-Filter Assembly 95 compares SED, DL and ROP predictions against each other. There are thresholds established using historic data where if the three predictions do not agree within the established thresholds, then the prediction is ignored. The thresholds are established using historic drill cone sampling and SED, ROP and DL for the same historic holes.

Example pseudo code for operation of the Correct-and-Filter Assembly 95 is as follows:
Set MAS Hardness Detection to FALSE IF
{
[(DL_{hardness}=Extra Hard) AND (SED_{hardness}!=Extra Hard OR ROP_{hardness}!=Extra Hard)] OR

[(DL_{hardness}=Hard) AND (SED_{hardness}=Medium OR SED_{hardness}=Soft OR ROP hardness Medium OR ROP hardness Soft)] OR
[(DL_{hardness}=Soft) AND (SED_{hardness}!=Soft OR ROP_{hardness}!=Soft)]

### Measure While Drilling (MWD) Data

As previously alluded to, and illustrated in FIG. 9, key measured process parameters that are used from Measure While Drilling are:
Rate of Penetration (ROP) data;
Force on Bit (FOB) data;
Rotation Speed data; and
Torque data.

There are additional derived process parameters including Specific Energy of Drilling (SED) (see Equation 2-1) which is an important process parameter for controlling the drilling procedure. The MWD data is aggregated by depth down the hole.

### Validation of Hardness and Establishment of Thresholds

When Resource Geologists perform Material Type logging for Resource Evaluation holes (Reverse Circulation, RC holes), the samples are typically logged at 2 m depth intervals. During this process, the Resource Geologists estimate the proportion of each material type that make up the log sample. These are used to predict the product grades, lump product fraction, density, material handling characteristics etc. This well-established process is used as input to plant design, mine plans and blast designs. Before mining commences, the Material Type logging is used to estimate the hardness of the deposit to estimate Processing Plant and Drill and Blast requirements.

For production drilling, Mine Geologist sample a selection of holes, for example every second hole in every second row, for confirmed not-waste by visually inspecting drill cuttings. For determining blast domains, the material type proportions are roughly used to estimate the hardness, i.e. the hardness that is estimated is not quantitatively based on the Material Types, but rather the Mine Geologist's knowledge of how hard a combination of materials would be. The relative hardness is typically used in the Blast Domain which is issued once the production drilling is complete. The intent is to inform Drill and Blast Engineers to help assist with charge design.

In order to validate hardness models, a comprehensive list of relative hardness for all Material Types across a mine site (504 different types) was used. Once the Material Types are identified by the Mine Geologist per production hole (~25% of not waste holes), the proportion of soft, medium and hard material types are summed to provide fraction of each per hole. The calculation for Material Type Hardness is to multiply the proportion of hard by 100 and add to the proportion of medium multiplied by 50. The purpose of this process is to provide single score of hardness per hole ranging from 0 to 100, where 0 indicates presence of all soft material and 100 indicates presence of all hard material in the hole.

### Material Type Hardness Distributions

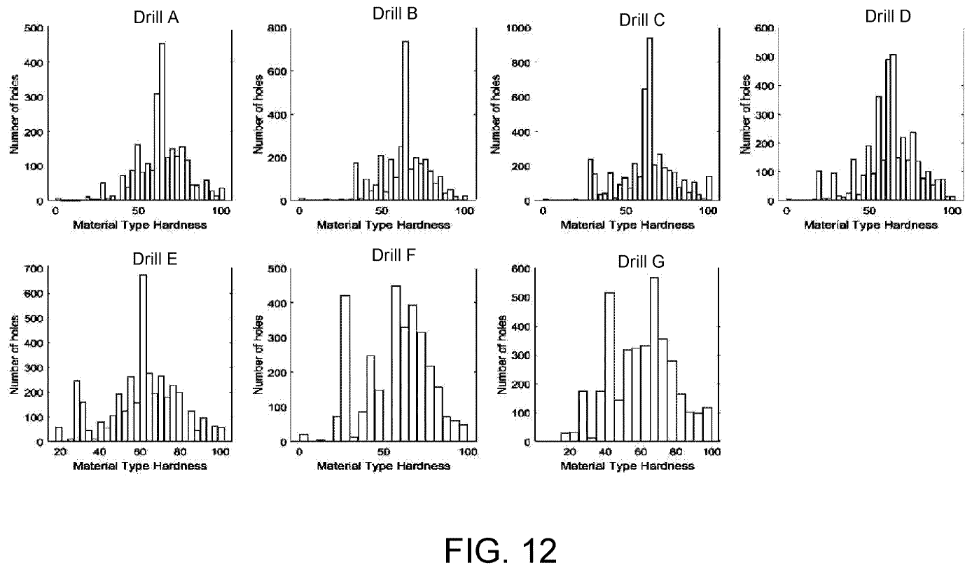

Histograms of Material Type hardness distribution per hole for each of seven autonomous drills A to G are shown in FIG. 12. The different drills are different models. The histograms of FIG. 12 illustrate that each of the drills drilled similar material during the analysis time window (2 years). Each histogram has little skew and a mode of around 60.

### MAS Hardness Distributions

It was found that Material Type Hardness, DL hardness predictions, SED and ROP are relatively normal distributions. This allows for Material Type data to be used as a benchmark to define the soft, medium and hard thresholds of each model.

Referring again to FIG. 9, the Deep Learning Model 89 receives historical data for threshold calibration 99. The primary reason for establishing thresholds is to identify a tipping point in the amount of soft, medium and hard section in a hole that justifies action. For example, if the hardness predictions are averaged per hole and if there is a small section of very hard rock, this data would be lost. This method instead marks that section of hole as hard (provided length of hard region is above the threshold).

The thresholding process involves:
Calculate p (high), SED and ROP for 0.1 m intervals.
Threshold the p (high), SED and ROP such that
   Model output>Threshold(hard) then the segment is hard
   Model output<Threshold(soft) then the segment is soft
For each half of hole, calculate the fraction of each segment such that:
   Fraction of segments that are hard>Percent (hard), then the half-hole is hard
   Fraction of segments that are hard>Percent (soft), then the half-hole is soft
   Remaining half-holes are medium

The top and bottom half hardness is written out to Top Hardness and Bottom Hardness fields and using lookup in Table 2 represented as total hole hardness. The primary reason for converting top and bottom hardness to single value is to assist with Change Management. The current practice is to charge a hole using single explosive product and as such top and bottom hardness is combined to a single value.

TABLE 2
Schema for Calculating Relative Hardness of Hole

| | | | | | | | | | |
|---|---|---|---|---|---|---|---|---|---|
| Top | Hard | Hard | Hard | Medium | Soft | Medium | Medium | Soft | Soft |
| Hardness | | | | | | | | | |
| Bottom | Hard | Medium | Soft | Hard | Hard | Medium | Soft | Medium | Soft |
| Hardness | | | | | | | | | |
| Hole | Very | Hard | Hard | Hard | Hard | Medium | Medium | Medium | Soft |
| | Hard | | | | | | | | |

It was found that the DL model provides the closest estimations to Material Type Hardness followed by ROP and SED.

It is important to note that benchmarking above is against an estimated data, not measured. Notwithstanding this, the proximity of loss functions and normal distributions across models justify the use of double thresholding method set thresholds for soft, medium and hard.

### Model Accuracy Comparison

It was found that there is not a clear correlation between Material Type, Hardness and the model hardness even when averaging large numbers of holes.

Material Type is prone to Mine Geologist interpretation. Models are prone to drilling inefficiencies (discussed in next section). For this reason, the model had to be proven through site-based Proof of Concept.

### Hardness Clustering

After the above-described thresholding process, the hole hardness classifications are geometrically clustered to smooth the data for the purpose of making it easier for operations to implement frequent change in charge design across the shot. The original purpose was that the hole charging process needs areas of the same explosive type for loading efficiency purposes. However, there is also the added benefit of removing outliers of hardness and of making it easier, more intuitive, and more reliable for the charge designs to be designed and reviewed.

### Limitations of Models and Edge Cases

Hardness predictions were tested against expected hardness by site. This process used Blast Domain data and Drill and Blast Engineer domain knowledge to compare ROP, SED and DL model predictions against expected hardness. Overall there was good alignment (for >90% of patterns, for 31 out of 34 semi-auto patterns) where if DL model predictions spatially represent similar regions of hardness levels when compared to average ROP and SED. Below section summarises scenarios where there was misalignment.

### Misalignment Between Drill Bit Type and Drill Configuration

MAS Drill system uses a customised operating mode for different drill bit types, for example bits are designed for different ground hardness's. While the overall target of maintaining a constant torque does not change, drilling inputs adjust and account for different characteristics of a drill bit. If there is a mismatch between drill bit diameter and pattern design, MAS Drill system stops operation and provides alert. However, such is not the case for other properties of the drill bit. The operators have control over selection of the drill bit and hence the corresponding operating configuration. If a different bit to that which is installed is selected, the hardness predictions provide incorrect and contradictory outputs.

The effect of incorrect drill configuration on the ROP model is overprediction of the ground hardness. This is because the Force on Bit (FOB) is not providing the calibrated force as required by the automated system leading to decreased ROP. The DL model is less susceptible to this as it accounts for the decreased FOB. Further, it is possible to train the DL for these type of scenarios by including such events in the training dataset. If D&B (drill and blast) Engineers make charging decisions based on an average ROP it will lead to incorrect charge design, in some cases significant undercharge and others significant overcharge.

### Drill Bit Model and Drill Bit Wear

The Inventors found that for a nominal hole diameter of 229 mm, approximately 10% of variance in the relationship between ROP and SED is attributable to drill bit. The inclusion of the drill bit in analysis increases the correlation of the relationship by ~5%. Additionally, when bit model is accounted in the inverse relationship between SED and thrust contribution of SED, some drill bit types exhibit variance to general relationship. Normally at higher energies (harder rock), the vertical thrust component of SED decreases as the angular energy contribution increase.

### Faulty or Uncalibrated Sensor\Hardware

For some of the sensor hardware such as rotation speed and FOB, MAS Drill system can detect out-of-calibration or faulty hardware by comparing the sensor data to expected data. In situations where rotation speed sensors had not been fitted the DL model was customised to omit rotation speed from inputs. However, this impacted the SED model.

For other hardware such as air nozzle, faulty hardware detection is more complex. If an air nozzle is faulty, it would not deliver enough air pressure to blow the drill cuttings out of the hole. This results in increased torque and as such MAS Drill would reduce the FOB resulting in decreased ROP. The net effect of this would be models incorrectly predicting harder ground.

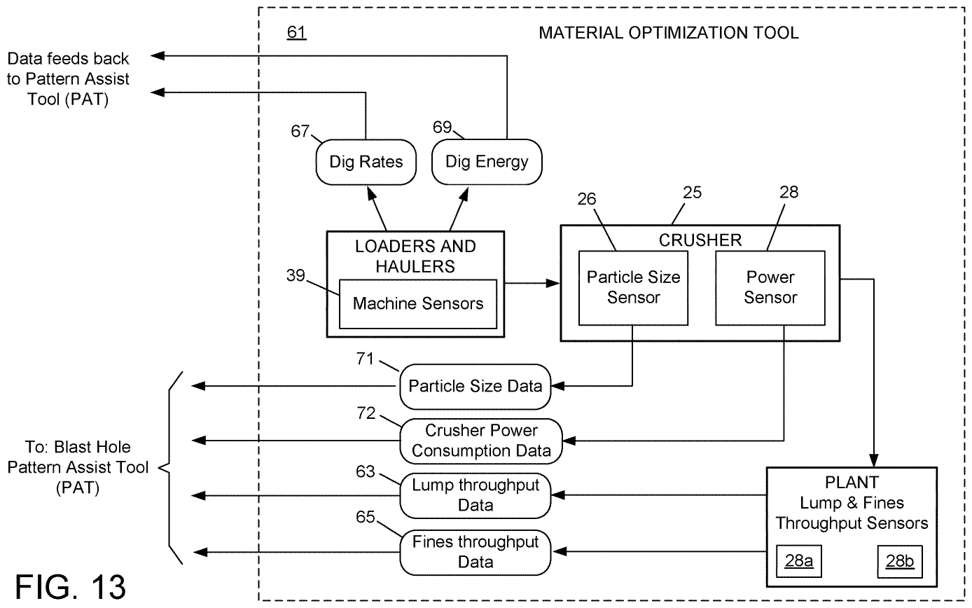

FIG. 13 is a detail of the Material Optimization Tool (MOT) 61a of FIG. 8. The MOT comprises a system of sensors that produces data 63, . . . , 72, from machines that dig crush and post-crush process, material from the most recently blasted bench. The Inventors have found that energy at the tooth 115 of a digger bucket 117, divided by payload, provides correlations between ground hardness and blast parameters. This energy may be termed Specific Dig Energy (SDE) or simply “Dig Energy” which the MOT 61a generates as Dig Energy data 69. Dig energy provides an indication of whether a correct amount of explosive was used and whether the blasting was timed correctly. Low dig energy may indicate that too much explosive material was used, which may have undesirably turned the rocks into dust, thereby losing “lump premium”.

The Inventors have sought correlations between parameters that are used in SDE, i.e. cylinder pressures of machines such as front end loaders, shovels, excavators etc, and ground hardness using a family of models generally referred to as Mining Automation System (MAS) Blast Predict Control (MBPC).

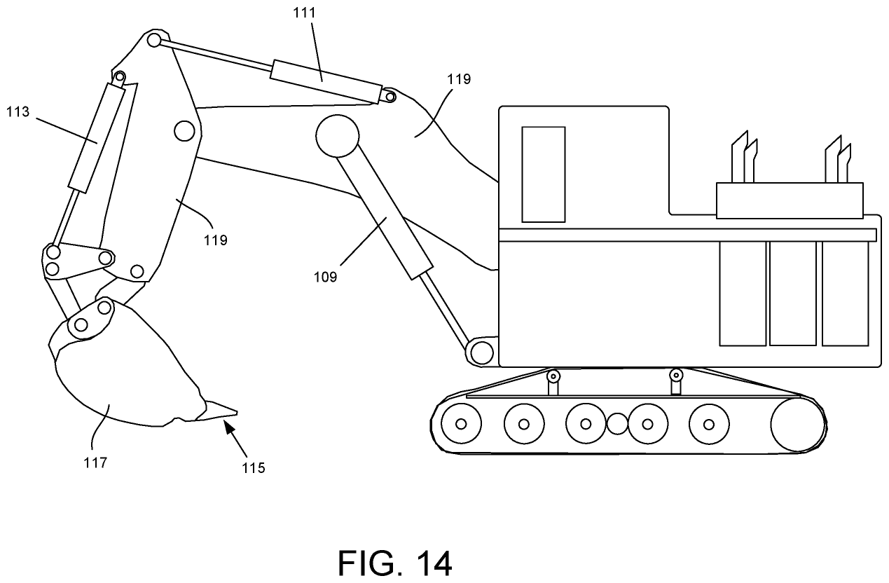

As indicated in FIG. 14, cylinder pressures were investigated for:
Boom retract cylinder 109;
Stick extend cylinder 111; and
Bucket extend cylinder 113.

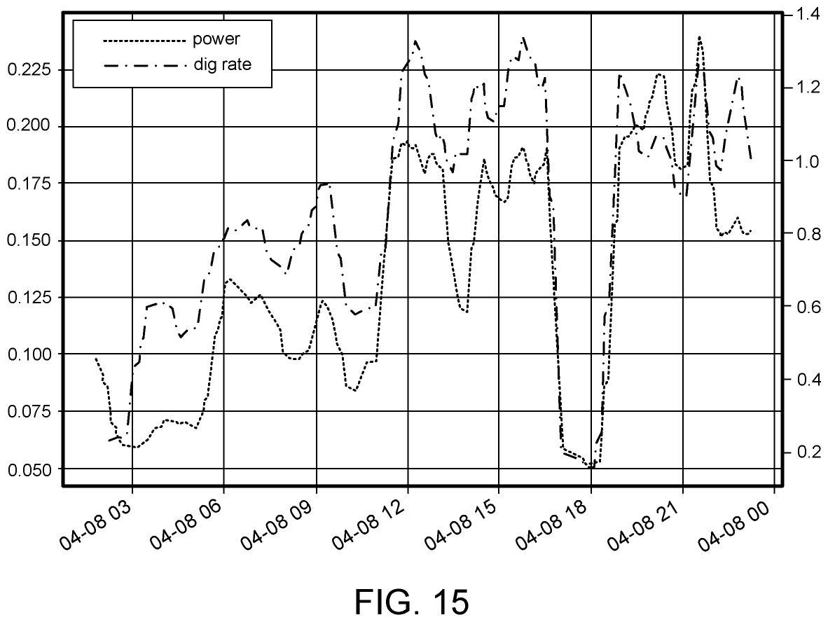

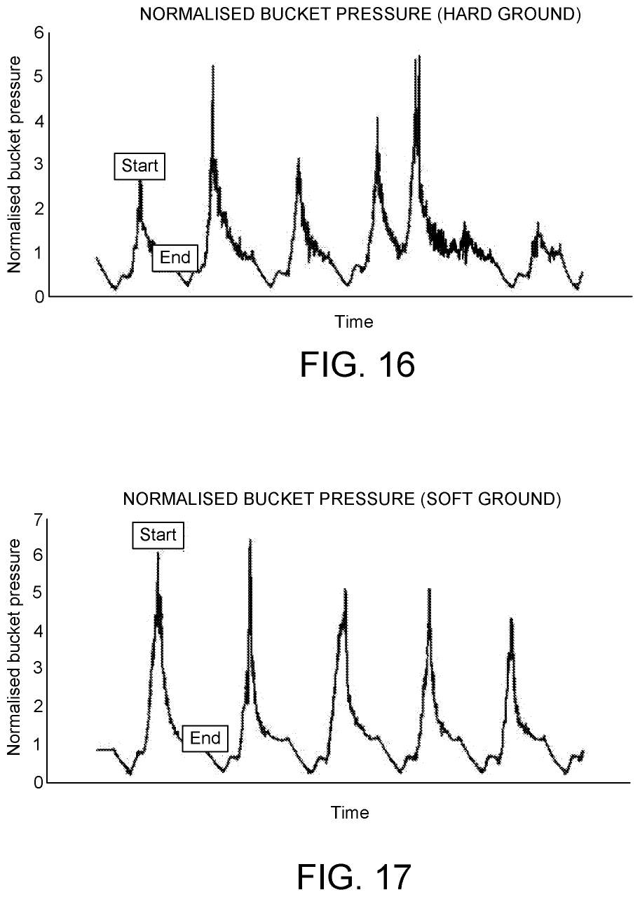

An increase in these pressures is associated with the digger's digging action, i.e., the movement of its arms 119, 121 and bucket 117 from the point of the bucket teeth 115 penetrating the ground to the teeth 115 subsequently being withdrawn clear of the ground. FIG. 15 is a graph illustrating the amount of power expended by the digger over a dig cycle compared to the dig rate. The Inventors' investigations led them to the following findings:
The type of digging that a digger performs is critical in the resulting associated dig energy. For example, batter work needs to be excluded if using it as feedback for blast performance (as blast parameters have little impact on energy required for batter work);
High boom retract cylinder pressures are associated with a slow dig rate and high bucket extend cylinder pressures are associated with a fast dig rate. It is believed that high boom retract-cylinder pressures are associated with how operators control the amount of force in hard ground and later is associated with well broken up ground that allows the operator to fill the bucket more;
There is “jerkiness” in the cylinder pressure signal when digging hard ground (FIG. 16) compared to a “smooth” pressure signal (FIG. 17) when digging soft ground;
Cylinder pressures can pin point problematic dig areas. For example, the Inventors observed a digging situation where there was a ~2 s period where a digger strained with a high cylinder pressure before it broke ground (after bucket angle change); and
That when digging soft ground, reduced efficiency can be observed due to a large amount of dust associated with the soft ground. This may be the reason for the higher-than-expected truck load times for soft ground.

Tables 3A and 3B set out correlation values between different values associated with dig energy (in the “Field Name” column) and each of a number of hardness prediction models. The various hardness prediction models are Hardness raw (deep learning), SED av (Specific Energy of Drilling) and ROP (Rate of Penetration) are different models of ground hardness and MBPC Hardness is a collation and clustering of these.

TABLE 3A

| | MBPC | MBPC | MBPC |
|---|---|---|---|
| Field Name | Hardness | Hardness raw | SED av |
| MBPC Specific Dig Energy | ↑0.39 | ↑0.33 | ↑0.34 |
| Filtered | | | |
| MBPC Specific Dig Energy | ↑0.38 | ↑0.31 | ↑0.32 |
| Normalised Filtered | | | |
| MBPC Normalised Duration | ↑0.20 | 0.13 | 0.14 |
| MBPC Dig Rate Normalised | −0.08 | 0.00 | 0.00 |

TABLE 3B

| | | | MBPC | MBPC |
|---|---|---|---|---|
| | MBPC | MBPC | Normalised | Dig Rate |
| Field Name | ROP av | delta | Duration | Normalised |
| MBPC Specific Dig Energy | ↓−0.35 | ↓−0.30 | ↑0.26 | −0.04 |
| Filtered | | | | |
| MBPC Specific Dig Energy | ↓−0.33 | ↓−0.29 | ↑0.28 | −0.05 |
| Normalised Filtered | | | | |
| MBPC Normalised Duration | −0.16 | −0.12 | 1.00 | −0.70 |
| MBPC Dig Rate Normalised | −0.01 | 0.01 | −0.70 | 1.00 |

MBPC delta attempts to model over-blasting (positive) and under-blasting (negative) by subtracting hardness from Effective Powder Factor. It had been expected that MBPC delta would yield the best correlation to dig energy, however the correlation turned out to be lower so that the Inventors decided that MBPC delta was not modelled accurately.

A new “dig-delta” model was to better estimate Dig Energy. This was done by solving for a, b and c using a Least Squares Estimate in the following equation to minimise error between actual dig energy and Dig Delta:

$$Dig Delta = {a}_{1} Powder Factor + {a}_{2} Explosive Mass + {a}_{3} Relative Weight Strength + {b}_{1} ( stem / burden ) + {b}_{2} ( spacing / burden ) + {b}_{3} ( depth / burden ) + {b}_{4} ( charge deck / burden ) + {b}_{5} ( explosive mass / burden ) + {c}_{1} hardness$$

The correlation between Dig Energy (actual) and Dig Delta (predicted) was found to be 0.44. While a 0.44 correlation may not be considered significant, the large amount of data from each blast hole and each dig cycle enable better spatial correlations.

It will be realised from the preceding discussion that there is provided a method to improve accuracy of hole-by-hole ground hardness predictions 59 (FIGS. 8, 9) for a bench such as Bench i+1 (item 124c, FIG. 3) that is below a current bench, Bench i (item 124b, FIG. 3). In a preferred embodiment the method includes generating measure while drilling (MWD) data 54 (FIGS. 8, 9) whilst performing blast hole drilling, for example with Autonomous Drilling Systems (ADS) 1a, . . . , 1n (FIG. 2) of the current bench. The MWD 54 data may include all the data set out in Table 1 (or more) but at least will include air pressure data, for example from the down hole air sensor 2a (FIG. 2), indicating air pressure measurements from a pressurized air delivery arrangement such as an air nozzle and torque data 54b (FIG. 9), which indicates measurements of torque applied by the ground being drilled to oppose rotation of the drill bit in performing the blast hole drilling. (The drill bit is visible below sensor 2a in FIG. 1.)

The method also includes determining the occurrence of fallback, or malfunction of the pressurized air delivery arrangement during drilling based upon the air pressure data and the torque data. As previously mentioned, fallback is a phenomenon associated with soft ground where the sides of the hole being drilled fall into the hole and block the pressurized air delivery arrangement, e.g., the nozzle. MWD collected during fallback, or an air nozzle malfunction, has been found to cause unreliable data. Accordingly, the method also includes discarding some or all MWD data collected during the occurrence of the fallback or malfunction of the air delivery arrangement, whilst generating the hole-by-hole ground hardness predictions.

It will also be realized from the preceding discussion that a method for determining a drill pattern for a bench, such as Bench i+1 (item 124c, FIG. 3), that is below a current bench, e.g. Bench i (item 124b, FIG. 3) has been described. The method for determining the BBDP involves generating measure while drilling (MWD) data 54 from blast hole drilling of the current bench, for example by use of automatic drilling systems 1a, . . . , 1n, according to a current bench drill pattern (CBDP). A hardness model 55a is provided that is responsive to the MWD data 54 of the current bench to thereby produce hole-by-hole ground hardness predictions 89a of the bench below. The BBDP is then determined, for example by pattern assist tool 73a (FIG. 8) based on at least the hole-by-hole ground hardness predictions 89. The current bench drill patterns are then updated as illustrated in box 75 (FIG. 8) to the BBDP prior to blast hole drilling the bench below.

The term “comprises” and its variations, such as “comprising” and “comprised of” is used throughout in an inclusive sense and not to the exclusion of any additional features. It is to be understood that the subject matter is not limited to specific features shown or described since the means herein described herein comprises preferred forms of putting the various embodiments and aspects into effect.

Throughout the specification and claims (if present), unless the context requires otherwise, the term “substantially” or “about” will be understood to not be limited to the value for the range qualified by the terms.

Any embodiment is meant to be illustrative only and is not meant to be limiting.

1. A method to improve accuracy of hole-by-hole ground hardness predictions for a bench (“bench-below”) that is below a current bench of an open cut mine, the method comprising:
generating measure while drilling (MWD) data, whilst performing blast hole drilling of the current bench, wherein the MWD data includes,
air pressure data indicating air pressure measurements from an air pressure sensor, and
torque data indicating measurements of torque opposing rotation of a drill bit performing the blast hole drilling;
determining occurrence of fallback or malfunction of a pressurized air delivery arrangement during drilling based upon the air pressure data and the torque data; and
discarding some or all MWD data collected during the occurrence of the fallback or malfunction of the pressurized air delivery arrangement, whilst generating the hole-by-hole ground hardness predictions.

2. The method of claim 1, wherein the determining of the occurrence of fallback or malfunction during drilling includes monitoring the air pressure data and the torque data to identify an increase in air pressure whilst at the same time identifying an increase in torque.

3. The method of claim 1, wherein the determining of the occurrence of fallback or malfunction of the pressurized air delivery arrangement during drilling is performed by a fallback model that is responsive to the air pressure data and the torque data, wherein the fallback model generates a fallback detected signal that indicates the occurrence of the fallback or malfunction of the pressurized air delivery arrangement.

4. The method of claim 1, wherein the method includes generating the ground hardness predictions by applying the MWD data to a pre-trained deep learning model wherein the pre-trained deep learning model is responsive to the MWD data to produce deep learning (DL) hardness prediction data.

5. The method of claim 4, including:
providing a Rate of Penetration (ROP) model that is responsive to ROP data of the MWD data, wherein the ROP model produces ROP hardness data indicating hardness of the current hole; or
providing a Specific Energy of Drilling (SED) model, wherein the SED model is configured to produce SED Hardness data in response to data of the MWD data.

6. The method of claim 4, including providing a Specific Energy of Drilling (SED) model, wherein the SED model is configured to produce SED Hardness data in response to data of the MWD data,
wherein the SED model is configured to produce the SED Hardness data in response to the torque data, RPM data, force on bit (FOB) data and ROP data of the MWD data or wherein the SED model is configured to produce the SED Hardness data in response to the torque data, RPM data, force on bit (FOB) data and ROP data of the MWD data and the model is the SED model is configured to take into account characteristics of the drill bit.

7.-8. (canceled)

9. The method of claim 5, including generating the ground hardness predictions by applying the deep learning hardness prediction to a Mining Automation System (MAS) Hardness Model.

10.-13. (canceled)

14. The method of claim 1, including providing a pattern assist tool (PAT) to generate a bench below drill pattern (BBDP) based on at least the hole-by-hole ground hardness predictions.

15. The method of claim 14, wherein the PAT is configured to produce the BBDP based on the hole-by-hole hardness predictions and one or more sensors of post-blast machines used to transport and process material blasted from the current bench or wherein the PAT is configured to produce the BBDP based on the hole-by-hole hardness predictions and one or more sensors of post-blast machines used to transport and process material blasted from the current bench and the method includes providing a Materials Optimization Tool (MOT) that includes one or more sensors of the post-blast machines.

16.-23. (canceled)

24. The method of claim 14, wherein the PAT is configured to take into account local geotechnical surfaces data when producing the bench below drill pattern.

25. The method of claim 14, including operating autonomous drilling systems (ADS) to execute a current bench drill pattern to perform the blast hole drilling where the current bench drill pattern is updated to the bench below drill pattern determined by the PAT subsequent to blasting, digging and crushing of the current bench.

26. The method of claim 1, including determining hole-by-hole charge specifications for holes in the current bench from the hole-by-hole ground hardness predictions.

27.-30. (canceled)

31. A method for determining a drill pattern for a bench (“bench below drill pattern” (BBDP)) that is below a current bench, the method comprising:
generating measure while drilling (MWD) data from blast hole drilling of the current bench according to a current bench drill pattern (CBDP);
providing a hardness model that is responsive to the MWD data of the current bench to thereby produce hole-by-hole ground hardness predictions of the current bench;
determining the BBDP by projecting the hole-by-hole ground hardness predictions of the current bench to the bench below; and
updating the CBDP to the BBDP prior to blast hole drilling the bench below.

32. The method of claim 31, wherein the hardness model comprises a deep learning (DL) model that is trained to generate deep learning hardness predictions (DL hardness predictions) from the MWD data.

33. The method of claim 31, wherein the hardness model includes a fallback model that is configured to monitor torque data and air pressure data of the MWD and to produce a fallback detect signal therefrom indicating presence of drill hole fallback or malfunction of a pressurized air delivery arrangement.

34. The method of claim 33, wherein the hardness model is responsive to the fallback model and is configured to discard one or more data making up the MWD data upon receiving the fallback detect signal.

35. The method of claim 32, including a rate of penetration (ROP) model that is responsive to ROP data of the MWD data, wherein the ROP model is configured to produce ROP hardness data indicating a hardness value at a position of the current bench.

36.-39. (canceled)

40. The method of claim 31, wherein determining the BBDP is further based on local geotechnical surfaces data.

41. The method of claim 31, wherein determining the BBDP is further based on data from sensors of a material optimization tool (MOT).

42. The method of claim 41, wherein the data from the MOT sensors includes one or more of:
particle size data indicating size of particles coming out of a crusher crushing blasted material of the current bench;
crusher power consumption data;
dig energy data, being data indicating energy required to dig the blasted material of the current bench;
dig rate data;
plant throughput data, being data from a plant throughput sensor for detecting material from a post-crusher material processing plant.

43.-47. (canceled)
